(19) United States
(12) Patent Application Publication
(10) Pub. No.: US 2006/0052307 A1
(43) Pub. Date: Mar. 9, 2006

(54) Bone growth factor

(76) Inventor: Cherk Shing Tam, Oakville (CA)

Correspondence Address: TORYS LLP, 79 WELLINGTON ST. WEST, SUITE 3000, TORONTO, ON, M5K 1N2, CA

(21) Appl. No.: 10/537,341

(22) PCT Filed: Dec. 5, 2003
(86) PCT No.: PCT/CA03/01898

§ 371 Date: Jun. 3, 2005

Related U.S. Application Data
(63) Continuation-in-part of application No. 60/430,932, filed on Dec. 5, 2002.

(51) Int. Cl. A61K 38/08; C07K 7/08

(52) U.S. Cl. 514/15; 530/326

(57) ABSTRACT

A compound having bone stimulatory activity: 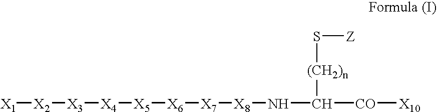 in which:
X₁ and X₁₀ are positively charged polar amino acids;
X₄ and X₈ are negatively charged polar amino acids;
X₅ is an aromatic amino acid;
X₂, X₃, X₆ and X₇ are non polar neutral amino acids or uncharged polar amino acids; Z represents a blocking group; and n is an integer from 1 to 3.

### FIELD OF INVENTION

The present invention relates to peptides which stimulate bone growth.

### BACKGROUND OF THE INVENTION

Understanding of issues related to bone growth and mechanical strength has progressed over the years, a summary being provided in international patent application No. PCT/CA 94/CA144 published under international publication No. WO94/20165 on Sep. 15, 1994.

Various approaches to the treatment of diseases involving reduction of bone mass or bone mineral density are exemplified in the patent literature.

Several patent publications of the present Applicant disclose various proteins and peptides having bone stimulatory effects. For instance, each of U.S. Pat. No. 6,117,839, issued Sep. 12, 2000, and WO 00/75185, published Dec. 14, 2000, describes peptides containing 10-amino acids with bone stimulatory activity.

### SUMMARY OF THE INVENTION

The present invention is based on Applicant's findings that modification of the sulfur group of the cysteine residue leads to an increase in the effectiveness of the corresponding decamer in stimulating bone growth.

Accordingly, in one aspect of this invention, there is provided a novel class of peptides with bone stimulatory activity comprising an amino acid sequence containing 10-amino acids of the following Formula I: 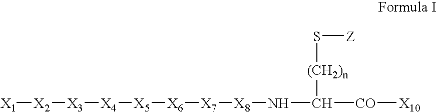
in which:
X₁ and X₁₀ are positively charged polar amino acids;
X₄ and X₈ are negatively charged polar amino acids;
X₅ is an aromatic amino acid;
X₂, X₃, X₆ and X₇ are non polar neutral amino acids or uncharged polar amino acids;
Z represents a blocking group; and n is an integer from 1 to 3.

In the context of this invention, “aromatic” amino acid includes phenylalanine, tyrosine, tryptophan, and histidine.

As a blocking group, Z is an alkyl group, carboxyalkyl or carboxyamidoalkyl group, or a substituted alkyl group including haloalkyl, alkenyl, alkynyl, cyclic, including carbocyclic and heterocyclic groups. The alkyl group (including those which are part of other groups, e.g., alkoxy) includes branched and unbranched alkyl groups with 1 to 12 carbon atoms, preferably 1 to 8 carbon atoms, most preferably 1 to 6 carbon atoms, especially 1 to 4 carbon atoms. Examples are: methyl, ethyl, propyl, butyl, pentyl, hexyl, etc. The terms propyl, butyl, pentyl or hexyl also include all the possible isomeric forms. For example, the term propyl also includes the two isomeric groups n-propyl and iso-propyl, the term butyl includes n-butyl, iso-butyl, sec. butyl and tert.-butyl, the term pentyl includes iso-pentyl, neopentyl, etc. Examples of haloalkyl groups are trifluoromethyl, trifluoromethoxy, difluoromethoxy, perfluoroethyl, perfluoropropyl, 2,2,2-trifluoroethyl, 2,2,2-trifluoroethoxy, 1,1,1-trifluoroprop-2-yl, etc. The term alkenyl group (including those which are part of other groups) includes branched and unbranched alkenyl groups having 2 to 12 carbon atoms, preferably 2 to 6 carbon atoms, particularly 2 to 4 carbon atoms, provided that they have at least one double bond, provided that they have at least one double bond, such as for example vinyl (provided that no unstable enamines or enolethers are formed), propenyl, iso-propenyl, butenyl, pentenyl and hexenyl. The term alkynyl groups (including those which are part of other groups) denotes alkynyl groups having 2 to 12 carbon atoms, preferably 2 to 6 carbon atoms, particularly 2 to 4 carbon atoms provided that they have at least one triple bond, e.g. ethynyl, propargyl, butynyl, pentynyl and hexynyl. The term halogen generally denotes fluorine, chlorine, bromine or iodine. A heterocyclic group contains nitrogen, oxygen and/or sulphur and can be an aromatic or saturated group having 5 or 6 cyclic atoms, wherein at least one cyclic atom is a heteroatom selected from among N, O and S, which may optionally be fused to another cyclic system.

Generally speaking peptides of the invention are synthetic peptides, being produced by conventional chemical or biotechnological methods. If found in nature, they are separated from cellular components with which they naturally occur, and referred to as isolated peptides.

Another aspect of this invention includes a method of stimulating bone growth in a mammal by administering an effective amount of such a decamer peptide to the mammal. The invention also includes a method of treating osteoporosis in a mammal by administering an effective amount of the peptide to a mammal. Typically, the mammal is a human.

The peptides can be administered prophylatically or therapeutically where the symptoms of the disease are already manifest. In terms of prophylactic treatment, a pharmaceutical of the present invention would typically be administered to an individual thought to be at an elevated risk of developing osteoporosis. Such a person could thus, for example, belong to one or more of the following groups of people: elderly people, particularly people over the age 60 years, post-menopausal women, and people whose lifestyles result in other risk factors, such factors including smoking, alcohol abuse, lack of exercise and low calcium intake. Genetic factors contributing to such susceptibility are also becoming better understood, as illustrated for example in U.S. Pat. No. 6,603,304, which issued Oct. 7, 2003.

The compounds or peptides of the present invention may also be used to treat a subject recovering from a trauma to the bone, such as a broken bone, in order to aid healing.

Desirably, the peptides of the invention are modified to include a “protecting” group at each end to reduce enzymatic or other degradation in the body.

As used herein, “protected” terminal amino group refers to a terminal amino group (N-terminus) coupled with any of various amino terminal protecting groups that can be employed in peptide synthesis. Examples of suitable groups include acyl protecting groups, for example, formyl, acetyl, benzoyl, trifluoroacetyl, succinyl, and methoxysuccinyl; aromatic urethane protecting groups, for example benzyloxycarbonyl; and aliphatic urethane protecting groups, for example t-butoxycarbonyl or adamantyloxycarbonyl (Gross and Mienhofer, eds., The Peptides, vol 3, pp. 3 to 88 (Academic Press, New York, 1981).

As used herein, “protected” terminal carboxyl group refers to a terminal carboxyl group (C-terminus) coupled with any of various carboxy-terminal protecting groups. As will be readily apparent to a person skilled in the art, suitable groups include amino, t-butyl, benzyl or other acceptable groups linked to the terminal carboxyl group through an ester or ether bond.

Peptides within the scope of this invention can be synthesized chemically by procedures well known in the art, such for example, solid phase peptide synthesis. The synthesis is commenced from the carboxy-terminal end of the peptide using an α-amino protected amino acid, t-butyloxy carbonyl (Boc) protective group, or any other suitable protective group.

Another aspect of the invention is a method for conducting a pharmaceutical business that includes (a) manufacturing a pharmaceutical preparation containing a compound of the invention, and (b) marketing to healthcare providers the benefits of using the preparation to stimulate bone growth, aid in the healing of damaged or broken bones, or to treat osteoporosis, including prophylactic treatments.

The invention includes a method for conducting a pharmaceutical business that includes providing a distribution network for selling a pharmaceutical preparation containing a compound of the invention, and providing instruction material to patients or physicians for using the preparation to stimulate bone growth, aid in the healing of damaged or broken bone, or to treat osteoporosis, including prophylactic treatments.

The invention includes a kit containing a pharmaceutical preparation of the invention in an amount sufficient to enhance bone growth in a mammal and instructions, written and/or pictorial, describing the use of the preparation for stimulating bone growth. Preferably, such a kit provides the preparation in a single dosage form.

### DESCRIPTION OF PREFERRED EMBODIMENTS

In preferred decamer amino acids of Formula I, amino acids X₁ and X₁₀ are arginine or lysine; X₂, X₃, X₆ and X₇ are independently threonine, valine, serine, alanine or glutamine, X₅ is histidine or phenylalanine; X₄ and X₈ are aspartic acid or glutamic acid; blocking group Z is an is an alkyl, carboxyalkyl or carboxyamidoalkyl group, and n is an integer form 1-3. More preferably, the alkyl residue in the Z blocking group is a lower alkyl group, such as methyl.

The presence of the blocking group Z on the sulfur atom prevents covalent dimerization of the peptides through formation of disulfide bridges while retaining the presence of the sulfur atom present in prior compounds known to have bone stimulatory activity. This provides decamer peptides with improved stability and surprising enhancement of biological activity.

In an embodiment of the invention, the decamer peptides of Formula I, contain methionine, a modified methionine or a modified cysteine at the 9-position of the amino acid sequence and are represented by Formula Ia below:
Ac-K T Q E F T A E X₉ K—NH₂
Ac-R T Q E F T A E X₉ K—NH₂
Ac-R T Q E H T A E X₉ K—NH₂
Ac-K T Q E H T A E X₉ K—NH₂ Formula Ia
in which X₉ is methionine or a modified methionine or modified cysteine. In the context of this invention, a “modified” cysteine is a cysteine residue of a peptide in which the hydrogen of the thiol group has been replaced by a covalently linked organic group that precludes dimerization of one cysteine with another by formation of an S—S linkage. The modification can be by means of a blocking group. Similarly, a “modified” methionine has its methyl group substituted by such an organic group.

In a particular embodiment, the modified methionine or cysteine is represented by the formula: 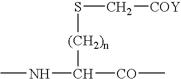
wherein Y represents a hydroxyl, alkoxy or amino group; and n is an integer form 1-3.

In a narrower embodiment of this invention, specific decamer peptides embraced by Formula Ia include the following decamers in which the ten amino acids have the sequence identified by the appropriate Sequence ID NO:

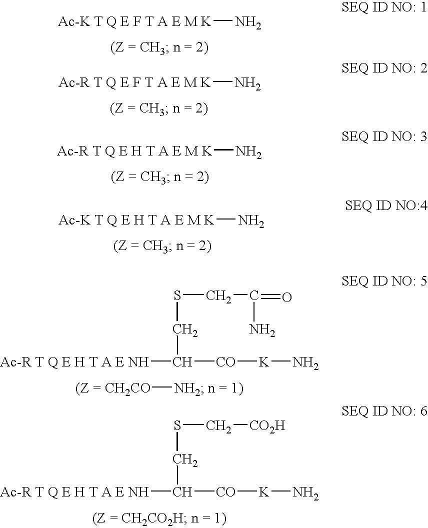

In the context of this invention, a peptide described as having a particular amino acid sequence includes a sequence in which the terminal amino acids are protected or unprotected, unless otherwise specified. Thus, for example, a peptide simply described as having the amino acid sequence of SEQ ID NO:1 has the sequence K-T-Q-E-F-T-A-E-M-K, and may or may not be protected.

### BRIEF DESCRIPTION OF THE DRAWINGS

Various objects, features and associated advantages of the invention will be more fully appreciated when considered in conjunction with the accompany Figures which are referred to in more detail in the examples presented below.

FIG. 1 illustrates an HPLC plot generated by the separation of the monomer and dimer of a peptide having the sequence Ac-R T Q E H T A E C K —NH₂ (SEQ ID NO:7). Optical density (left-hand axis; 212 nm) and percent acetonitrile (right-hand axis) are shown as a function of retention time (minutes) during separation of the monomer and dimer of the peptide having SEQ ID NO: 7 using reverse phase HPLC. The second and third peaks represent the monomer and dimer, respectively. The sample was dissolved in 10 μl of 0.1% trifluoroacetic acid and run in a continuous gradient at a flow rate of 0.9 mL/minute. Solvent A was 0.1% TFA and solvent B was acetonitrile with 0.1% TFA. The gradient slope was from 5% A to 14% B in 12 minutes.

FIG. 2 shows the effect of dimerization on a peptide having SEQ ID NO:7 on bone mineral apposition rate. Bone mineral apposition rate (μM/day) is shown on the vertical axis. The first bar is control, the second bar is dimer, and the third bar is monomer (N=4).

FIG. 3 illustrates the bone mineral apposition rate (μM/day) for representative peptides provided by the peptides identified as SEQ ID NO: 2 (Bar A), SEQ ID NO:1 (Bar B), SEQ ID NO:3 (Bar C), and SEQ ID NO:4 (Bar D), compared to a control (Bar E).

FIG. 4 illustrates the bone mineral apposition rate (μM/day) for a representative peptide provided by the invention with SEQ ID NO: 2 (Bar D), compared to a control (Bar A) and two peptides in a monomer and dimer forms, SEQ ID NO: 7 (Bar B, monomer; Bar C, dimer) and Ac-R T N E H T A D C K —NH₂ (SEQ ID NO:8; Bar E, monomer; Bar F, dimer).

FIG. 5 illustrates the bone mineral apposition rate (μM/day) for a representative peptide of the invention with SEQ ID NO:5 (Bar A), compared to a control (Bar C), and the peptide identified as SEQ ID NO:7, in monomer form (Bar B).

FIG. 6 illustrates the bone mineral apposition rate (μM/day) for a representative peptide provided by the invention with SEQ ID NO:2 for male (●) and female (▪) rats. Dosages indicated along the abscissa are in nmoles/kg b.w. A single subcutaneous injection was given to each animal.

FIG. 7 illustrates the time-related response of bone mineral apposition (μM/day) for a representative peptide provided by the invention with SEQ ID NO:2 (▪) compared to a control (●). The arrow (day 0) indicates the point of administration of the peptide.

FIGS. 8(a), 8(b) and 8(c) illustrate the stability of various peptides of the invention compared to a prior art peptide over time under various conditions. The vertical axis shows percentage of the intact peptide solution at pH 7.4. The abscissa shows months. FIG. 5(a) is at 37 C; FIG. 5(b) is at room temperature; and FIG. 5(c) is a 4° C. The peptides evaluated corresponded to SEQ ID NO: 2 (□), SEQ ID NO:3 (Δ), SEQ ID NO:4 (X), and SEQ ID NO: 7 (●).

FIG. 9 illustrates the percentage change over time in the ratio of bone mineral content to bone weight (BMC/BW) in a series of in vivo experiments using a rat model and various peptides.

### EXAMPLES

The invention is further illustrated by the following non-limitative examples, in which the peptides used are described above and by their sequence identifiers.

General Comments

All the peptides were synthesized using solid state synthesis according to conventional procedures well known to those skilled in the art. All of the peptides used in the examples were acetylated at the amino terminus and amidated at the carboxyl terminus.

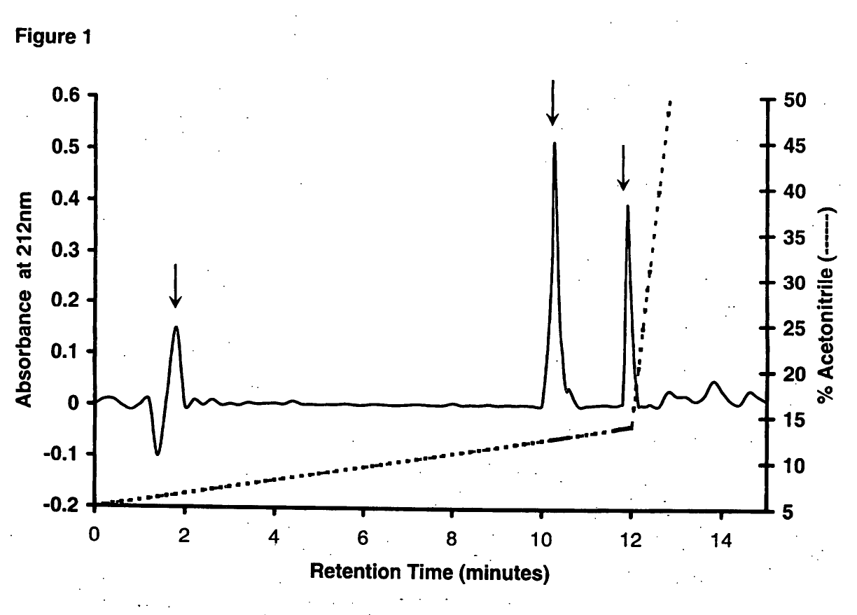

The peptides were purified to a purity above 95%. The purified form of the peptide having SEQ ID NO:7 was allowed to dimerize at room temperature in a solution of 1.7M ammonium sulfate and 50 mM sodium phosphate at pH 7.4. The dimerized form of the peptide having SEQ ID NO:7 was separated from the undimerized material by reverse phase HPLC using a Vydec semi-prep C18 column. The HPLC profile is shown in FIG. 1.

Test peptides were administered by subcutaneous injection to the right gluteal region of the rats after being dissolved in a carrier buffer comprising PBS containing 0.1% heat inactivated BSA at pH 7.4. The dose was 300 nmoles/kg bodyweight in an injection volume of 400 μl.

Male Sprague-Dawley rats from Charles River Laboratory were used. The weight of the animals was approximately 350 g. They were housed in pairs in standard shoebox cages. Purina rat chow and tap water were provided ad libitum. The light/dark cycle was 12 hours, the temperature 22° C. and the humidity 50%. The animals were acclimatized for one week before initiating dosing.

Bone mineral apposition rate as measured for the distal metaphysis of the right femur was the parameter for the assessment of activity. Two doses of tetracycline hydrochloride (Sigma), 24 mg/kg body weight were administered I.M. in an injection volume of 800 μl to the left gluteus maximus muscle. The first dose was given immediately following the administration of the peptide, and the second dose 48 hours later. 24 hours after the second dose of tetracycline, the animals were sacrificed by carbon dioxide narcosis. The right femur was dissected clear of muscles and a 0.5 cm cross section of the bone 1 mm below the upper epiphyseal cartilage was taken and processed undecalcified for histological preparation of 8 μM. Bone mineral apposition rate was measured in 15 randomly selected bone formation sites with two tetracycline labels in the trabecular bone. The arithmetic mean of these 15 sites was taken as the mean bone mineral apposition for the respective animals.

### Example 1

Effect of Dimerization on Activity of the Peptide having SEQ ID NO: 7.

Grouping of Animals:

Group A (4 rats)
Control receiving carrier buffer
Group B (4 rats)
Peptide having SEQ ID NO:7, dimer
Group C (4 rats)
Peptide having SEQ ID NO:7, monomer

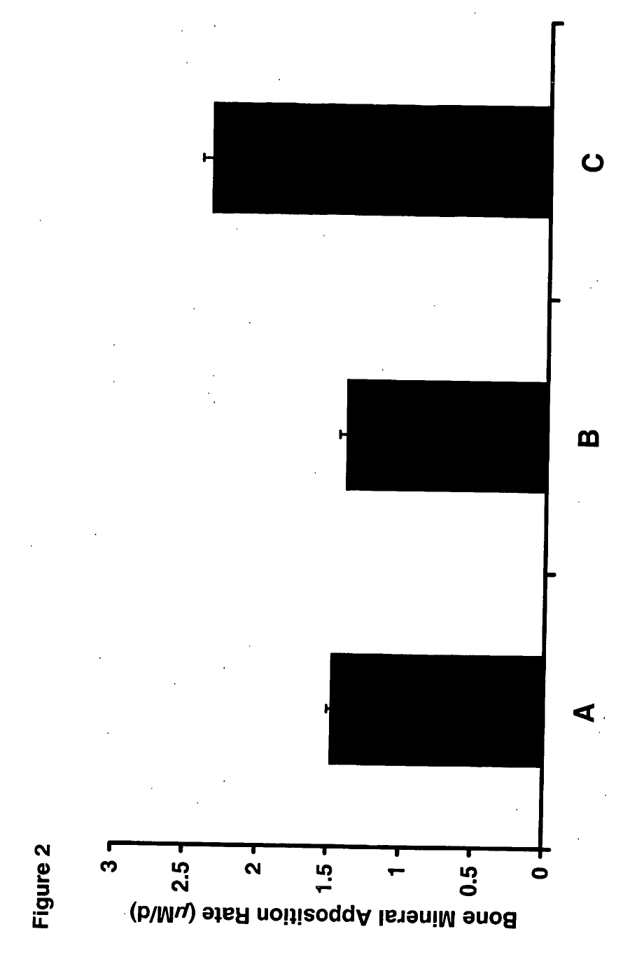

Table 1 shows the body weight of the rats. No significant difference in weight was found among the three groups of rats. The bone mineral apposition rate data are presented in Table 2 and FIG. 2. Somewhat surprisingly, the dimer form of the peptide demonstrated an inhibitory effect on bone mineral apposition rate. The bone apposition rate of this group was lower than that of the control group. However, the bone mineral apposition rate of the monomer treated groups was significantly higher than the control group as well as the group treated with the dimer.

TABLE 1
Body weight of Experimental Rats

| | | |
|---|---|---|
| | Group B | Group C |
| Group A | (SEQ ID NO: | (SEQ ID NO: |
| (control) | 7 dimer) | 7 monomer) |

| | | | | | |
|---|---|---|---|---|---|
| Rat 1 | 334 g | Rat 7 | 324 g | Rat 15 | 324 g |
| Rat 2 | 320 g | Rat 8 | 328 g | Rat 16 | 328 g |
| Rat 3 | 326 g | Rat 20 | 352 g | Rat 18 | 368 g |
| Rat 5 | 362 g | Rat 21 | 322 g | Rat 19 | 356 g |
| Mean | 335 g | | 334 g | | 344 g |
| (S.E.) | (9) | | (7) | | (11) |

TABLE 2
Bone mineral apposition rate (μM/d) of control rats and rats treated
with dimer and monomer of the peptide having SEQ ID NO: 7

| | | |
|---|---|---|
| | Group B | Group C |
| Group A | (SEQ ID NO: | (SEQ ID NO: |
| (control) | 7, dimer) | 7, monomer) |

| | | | | | |
|---|---|---|---|---|---|
| Rat 1 | 1.44 | Rat 7 | 1.50 | Rat 15 | 2.25 |
| Rat 2 | 1.53 | Rat 8 | 1.38 | Rat 16 | 2.24 |
| Rat 3 | 1.40 | Rat 20 | 1.28 | Rat 18 | 2.41 |
| Rat 5 | 1.53 | Rat 21 | 1.41 | Rat 19 | 2.54 |
| Mean | 1.48 | | 1.39 | | 2.36 |
| (S.E.) | (0.03) | | (0.05) | | (0.07) |

Dimerization of the peptide having SEQ ID NO:7 through disulfide bonding between the cysteine residues significantly reduced the stimulatory effect of the peptide on bone mineral apposition.

### Example 2

The Effect of Various Peptides on Bone Mineral Apposition Rate

Fifteen experimental rats were grouped into the following groups as follows:

| Group | Rat No. | Body weight (g) | SEQ ID NO |
|---|---|---|---|
| A | 1 | 558 | 2 |
| | 2 | 463 | 2 |
| | 3 | 558 | 2 |
| B | 4 | 543 | 1 |
| | 5 | 560 | 1 |
| | 6 | 550 | 1 |
| C | 7 | 543 | 3 |
| | 8 | 498 | 3 |
| | 9 | 510 | 3 |
| D | 10 | 551 | 4 |
| | 11 | 548 | 4 |
| | 12 | 483 | 4 |
| E | 16 | 580 | Control |
| | 17 | 513 | Control |
| | 18 | 521 | Control |

The peptides were prepared by dissolving the peptides in PBS with 0.1% heat inactivated BSA.

| SEQ ID NO | M.W. | Weight | Volume of buffer |
|---|---|---|---|
| 1 | 1280 | 0.7 mg | 1.378 ml |
| 2 | 1271 | 0.7 mg | 2.470 ml |
| 3 | 1243 | 1.0 mg | 1.947 ml |
| 4 | 1253 | 1.2 mg | 2.214 ml |

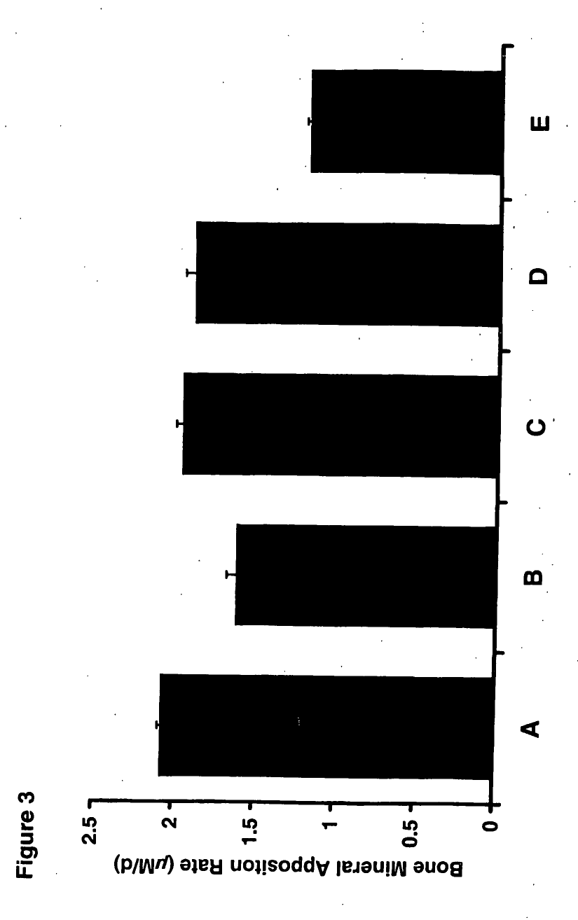

Table 3 below shows the average body weight for the respective groups. There was no significant difference in body weight among the five groups in this study. The mean bone mineral apposition rate for the respective groups is summarized in Table 4 and illustrated in FIG. 3. The bone mineral apposition rates of all peptides are significantly different from the control group.

TABLE 3
Rat Body Weights

| Group | Mean B.W. (g) | S.E. | N |
|---|---|---|---|
| A | 526 | 32 | 3 |
| B | 551 | 5 | 3 |
| C | 517 | 13 | 3 |
| D | 527 | 22 | 3 |
| E | 538 | 21 | 3 |

TABLE 4
Bone Mineral Apposition Rate

| Group | Mean rate (μM/d) | S.E. | N |
|---|---|---|---|
| A | 2.07 | 0.02 | 3 |
| B | 1.61 | 0.06 | 3 |
| C | 1.95 | 0.04 | 3 |
| D | 1.88 | 0.06 | 3 |
| E | 1.18 | 0.02 | 3 |

As can be seen from FIG. 3, the replacement of cysteine by methionine does not eliminate the stimulatory effect of the peptide on bone mineral apposition. The replacement of arginine by lysine at the N-terminus results in reduced activity. This substitution may render the peptide more susceptible to degradation by lysyl peptidase. The charge of the histidine residue does not appear to be essential for the activity of the peptide, as its replacement by the non-charged phenylalanine residue does not appreciably affect activity.

### Example 3

Comparison Between the Activity of the Peptide Having SEQ ID NO:2 and that of the Monomer and Dimer of the Peptides having SEQ ID NOs:7 and 8

The materials tested included the monomer and dimer of the peptide with having SEQ ID NO:7 and the monomer and dimer of the peptide with SEQ ID NO: 8: and the peptide with SEQ ID NO:2. These test peptides were administered in equal doses of 300 nmoles/kg body weight S.C. The testing procedure was the same as that used in the previous examples.

Twenty four male Sprague-Dawley rats were grouped into 6 age and weight matched groups. These 6 groups were subject to various treatments as following.

| | | |
|---|---|---|
| Group A | control, receiving carrier buffer | 4 rats |
| Group B | receiving SEQ ID NO: 7, monomer | 4 rats |
| Group C | receiving SEQ ID NO: 7, dimer | 4 rats |
| Group D | receiving SEQ ID NO: 2 | 4 rats |
| Group E | receiving SEQ ID NO: 8, monomer | 4 rats |
| Group F | receiving SEQ ID NO: 8, dimer | 4 rats |

The body weight of the six groups of rats is listed in Table 5.

TABLE 5
Body weights of experimental rats

| Group A | | Group B | | Group C | | Group D | | Group E | | Group F | |
|---|---|---|---|---|---|---|---|---|---|---|---|
| Rat # | wt (g) | Rat # | wt (g) | Rat # | wt (g) | Rat # | wt (g) | Rat # | wt (g) | Rat # | wt (g) |
| 10 | 312 | 2 | 312 | 3 | 320 | 14 | 310 | 9 | 292 | 16 | 314 |
| 17 | 320 | 7 | 306 | 8 | 290 | 18 | 306 | 13 | 320 | 20 | 328 |
| 25 | 308 | 11 | 320 | 15 | 322 | 24 | 312 | 22 | 318 | 23 | 298 |
| 29 | 312 | 12 | 314 | 21 | 320 | 28 | 322 | 30 | 318 | 26 | 312 |
| Mean | 313 | | 313 | | 313 | | 313 | | 312 | | 313 |
| (S.E.) | (3) | | (3) | | (8) | | (3) | | (7) | | (6) |

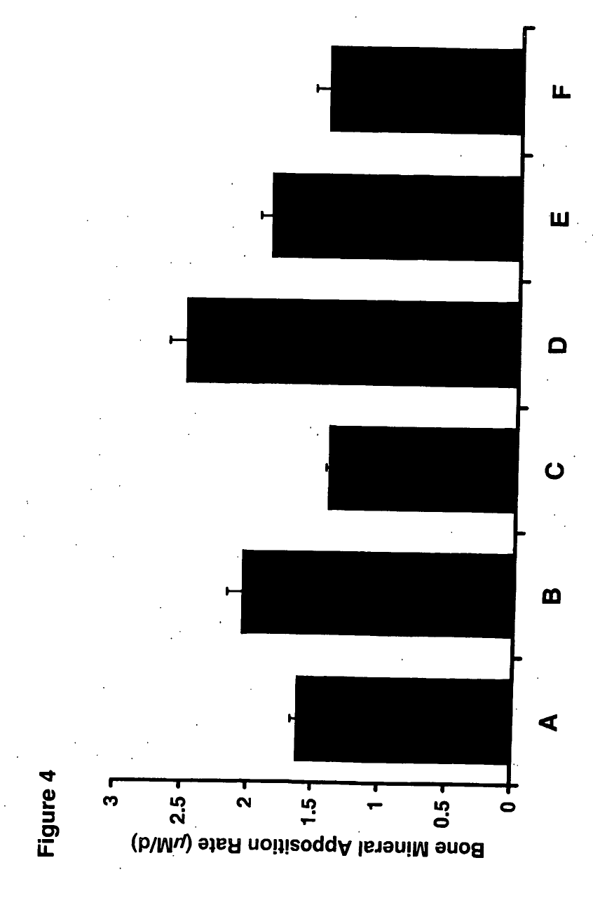

The effect of the various peptides on bone mineral apposition rate is summarized in Table 6 and illustrated in FIG. 4.

TABLE 6
Bone Mineral Apposition Rates of Rats Administered Different Peptides
and a Control.

| | | BONE MINERAL | |
|---|---|---|---|
| GROUP | RAT No | APPOSITION (μM/d) | MEAN |
| A | 10 | 1.78 | 1.62 |
| | 17 | 1.57 | |
| | 25 | 1.58 | |
| | 29 | 1.55 | |
| B | 2 | 1.91 | 1.86 |
| | 7 | 1.73 | |
| | 11 | 1.73 | |
| | 12 | 2.06 | |
| C | 3 | 1.29 | 1.43 |
| | 15 | 1.51 | |
| | 21 | 1.51 | |
| D | 14 | 2.25 | 2.49 |
| | 18 | 2.33 | |
| | 24 | 2.68 | |
| | 28 | 2.70 | |
| E | 16 | 2.27 | 2.05 |
| | 20 | 1.78 | |
| | 23 | 2.15 | |
| | 26 | 2.01 | |
| F | 9 | 1.39 | 1.40 |
| | 13 | 1.43 | |
| | 22 | 1.42 | |
| | 30 | 1.36 | |

The bone mineral apposition is significantly stimulated by monomers having the amino acid sequences identified as SEQ ID NOs:7 (Bar B) and 8 (Bar E). The dimers of these two peptides (Bars C and F, respectively) were found to significantly inhibit bone mineral apposition. The replacement of cysteine with methionine, as in SEQ ID NO:2, enhanced the stimulatory activity of the peptide (Bar D).

### Example 4

Comparison Between the Activity of the Peptide with SEQ ID NO: 5 and that of a Prior Art Peptide with SEQ ID NO:7

The peptide of SEQ ID NO:5, has a carboxyamidomethyl residue modifying cysteine at the 9-position of the decamer. This modified peptide was prepared in the following manner.

8.2 Mg of the peptide of SEQ ID NO:7 was dissolved in 1.5 ml. 0.1 M ammonium bicarbonate by slowly stirring in a water bath to maintain the temperature between 0° and 4° C. (Solution A). 62.2 Mg. of iodoacetamide (I CH₂ CO N H₂) was added to 0.8 ml. 0.1M ammonium bicarbonate (Solution B). 0.2 Ml of Solution B was added to Solution A. The mixture was stirred initially for 1 hr in a water bath maintained at 0° C. to 4° C., then stirred at room temperature overnight. The reaction mixture was injected directly into HPLC and purified to >85% purity.

Twelve male rats with a mass body weight of 300 g were divided into three groups of four rats. The test peptides (one to each group) were administered subcutaneously in equal doses of 300 nmoles/kg body weight. The procedure followed was the same as that employed in Examples 2 and 3.

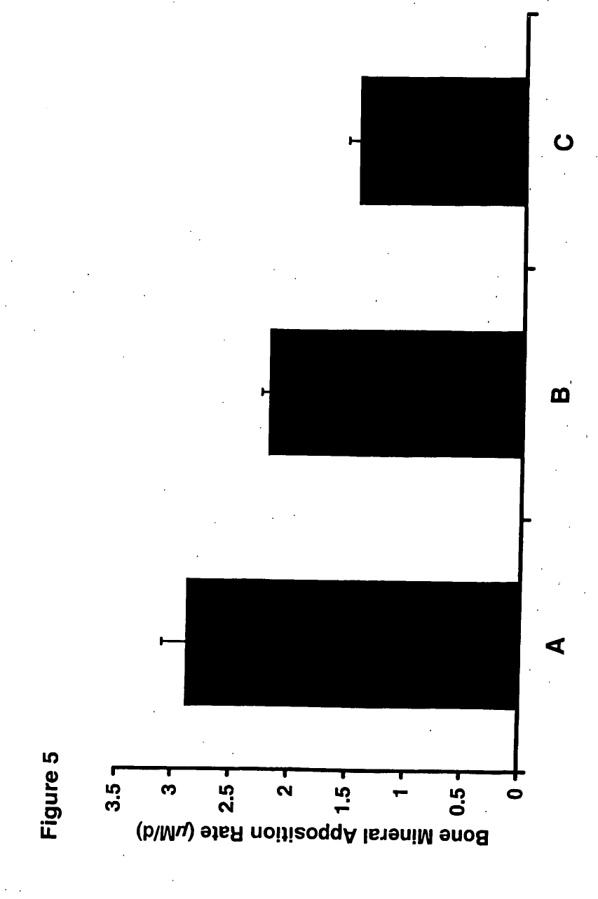

The effect of the peptides, compared to each other and to a control (Bar C), is illustrated in FIG. 5. As shown in the Figure, the bone mineral apposition rate of the modified peptide of SEQ ID NO: 5 (Bar A) in accordance with the invention was significantly greater than the prior art peptide of SEQ ID NO: 7 (Bar B).

Another modified peptide, that of SEQ ID NO:6 with a carboxymethyl residue modifying cysteine at the 9-position of the decamer, may be used to give similar results. This modified peptide is prepared by reconstituting 1 mg of the peptide with SEQ ID NO: 7 in 1 ml of 100 mM this pH 8.5. 1M iodoacetic acid is added with stirring and the mixture allowed to stand for 30 minutes at ambient temperature in the dark. 40 μl 1M dithiothreitol are added to quench the iodoacetic acid.

The reaction mixture is purified by HPLC and identity of the carboxymethylated peptide of SEQ ID NO:6 confirmed by mass spectroscopy.

### Example 5

Dose Dependent Effect of SEQ ID NO:2 on Bone Mineral Apposition

Experimental animals: Male Sprague-Dawley rats from Charles River Laboratory. Female Sprague-Dawley rats were bred at the animal facility of Erindale Campus, University of Toronto. All animals were approximately 3 months of age.

Animal grouping: The experimental animals were grouped according to sex and the dose of the peptide of SEQ ID NO:2 administered. The group mean of the body weight is shown in the following tables:

### Male Rats

| Group | Mean body weight (g) | S.E. | N | Dose of SEQ ID NO: 2 |
|---|---|---|---|---|
| A | 316 | 4 | 4 | 0 nmoles/kg |
| B | 315 | 7 | 4 | 50 nmoles/kg |
| C | 313 | 6 | 4 | 100 nmoles/kg |
| D | 313 | 6 | 4 | 200 nmoles/kg |
| E | 320 | 10 | 4 | 400 nmoles/kg |
| F | 323 | 9 | 4 | 800 nmoles/kg |

### Female Rats

| Group | Mean body weight (g) | S.E. | N | Dose of SEQ ID NO: 2 |
|---|---|---|---|---|
| G | 248 | 11 | 4 | 0 nmoles/kg |
| H | 239 | 9 | 4 | 50 nmoles/kg |
| I | 243 | 9 | 4 | 100 nmoles/kg |
| J | 247 | 12 | 4 | 200 nmoles/kg |
| K | 249 | 5 | 4 | 400 nmoles/kg |
| L | 245 | 6 | 4 | 800 nmoles/kg |

The dose of peptide was dissolved in 400 μl of buffer comprising of PBS with 0.1% of heat inactivated BSA and the method for the determination of bone mineral apposition rate was as described before.

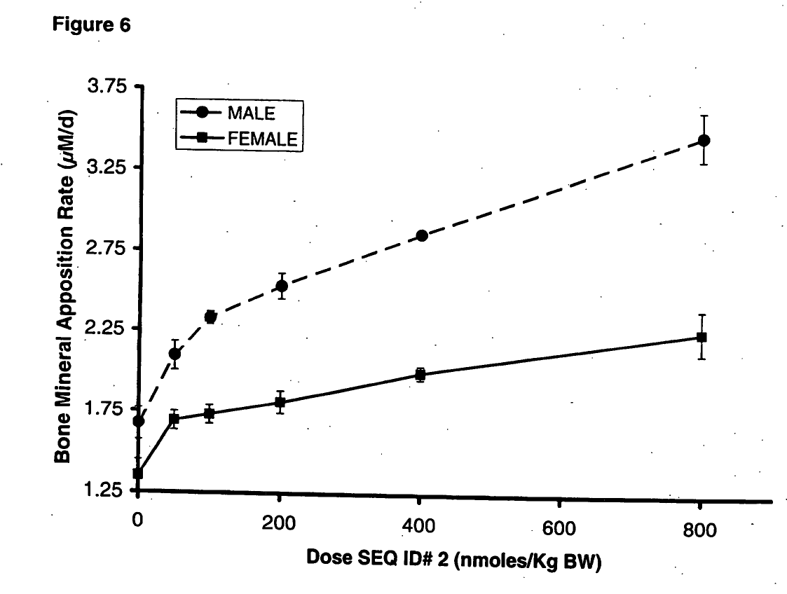

There was no difference in body weight among groups for the respective sexes. Male rats showed a significantly higher bone mineral apposition rats than the female rats of approximately the same age. In both male and female rats, there was a significant positive dose dependent response to the peptide having SEQ ID NO:2 as summarized in Tables 7 and 8, and illustrated in FIG. 6.

TABLE 7
Dose dependent response of bone mineral apposition to
SEQ ID NO: 2 in male rats

| Group | Dose of Peptide (nmoles/kg) | Mean rate (μM/d) | S.E. | N |
|---|---|---|---|---|
| A | 0 | 1.67 | 0.10 | 4 |
| B | 50 | 2.09 | 0.09 | 4 |
| C | 100 | 2.33 | 0.04 | 4 |
| D | 200 | 2.53 | 0.08 | 4 |
| E | 400 | 2.86 | 0.02 | 4 |
| F | 800 | 3.46 | 0.15 | 4 |

TABLE 8
Dose dependent response of bone mineral apposition to
SEQ ID NO: 2 in female rats

| Group | Dose of Peptide (nmoles/kg) | Mean rate (μM/d) | S.E. | N |
|---|---|---|---|---|
| G | 0 | 1.35 | 0.10 | 4 |
| H | 50 | 1.69 | 0.06 | 4 |
| I | 100 | 1.73 | 1.06 | 4 |
| J | 200 | 1.81 | 1.07 | 4 |
| K | 400 | 1.99 | 0.04 | 4 |
| L | 800 | 2.25 | 0.14 | 4 |

### Example 6

Time Related Response of Bone Mineral Apposition to a Single Administration of the Peptide having SEQ ID NO:2

Experimental animals. Male Sprague-Dawley rats from Charles River Laboratory. The age of the animals was approximately 3 months.

Experimental design: Animals were grouped in four. All groups received a single dose of SEQ ID NO:2 (300 nmoles/kg) subcutaneously injected to the right gluteal region at Day 0 at approximately the same time within 15 minutes. Two doses tetracycline labeling at intervals of 48 hours was used to measure bone mineral apposition rate. The labeling schedule starts at various time point for the respective groups as shown in Table 9. The control group received buffer injection only.

TABLE 9
Tetracycline labeling schedule

| Group | Day-1 | Day 0 | Day 1 | Day 2 | Day 3 | Day 4 | Day 5 | Day 6 | Day 7 |
|---|---|---|---|---|---|---|---|---|---|
| A | T | P | T | K | | | | | |
| B | | PT | | T | K | | | | |
| C | | P | T | | T | K | | | |
| D | | P | | T | | T | K | | |
| E | | P | | | T | | T | K | |
| F | | P | | | | T | | T | K |
| G | T | | T | K | | | | | |
| H | | T | | T | K | | | | |
| I | | | T | | T | K | | | |
| J | | | | T | | T | K | | |
| K | | | | | T | | T | K | |
| L | | | | | | T | | T | K |

P = SEQ ID NO: 2 administration
T = Tetracycline label
K = sacrifice

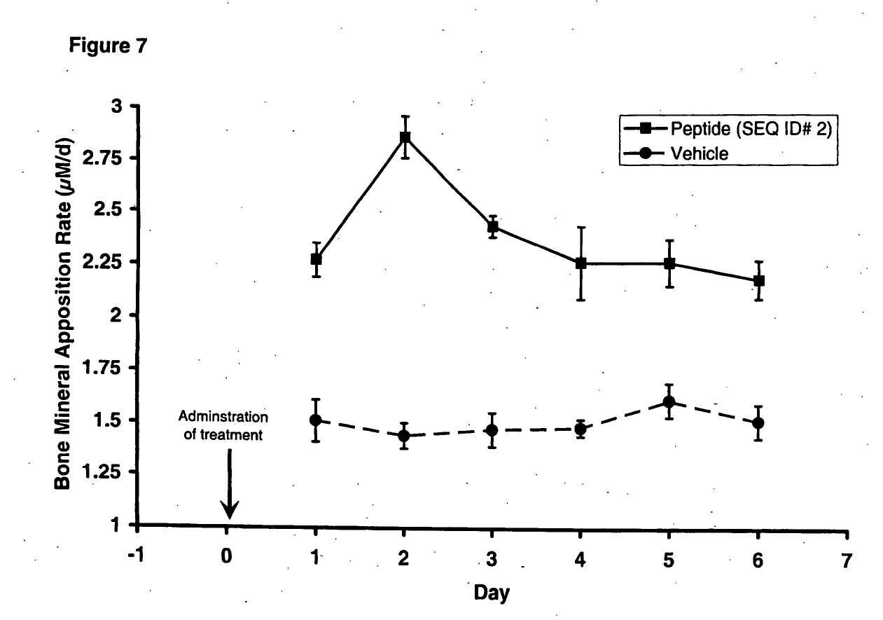

The changes in bone mineral apposition rate with time following a single dose of the peptide having SEQ D NO:2 are shown in FIG. 7. The stimulatory effect of the peptide on bone mineral apposition appears very early, within the first day. This stimulatory effect was sustained. The rate remains significantly higher than control even on the 6th day following a single injection. The data suggest that a peptide having SEQ ID NO:2 may be effectively administered at less frequent intervals than daily dosing.

The substitution of the cysteine residue with methionine prevents any significant dimerization and appears to be capable of enhancing the activity of the prior art peptide having SEQ ID NO:7. It appears that the charge of the histidine residue (residue No. 5) is not crucial for activity since it can be substituted with phenylalanine without influencing the effect on bone mineral apposition.

The long lasting stimulatory effect of the peptide having the amino acid of sequence identified as SEQ ID NO:2, following one single subcutaneous injection suggest that less frequent administration than the daily doses may be affective to stimulate bone growth.

### Example 7

Stability of Peptides of the Invention Compared to a Prior Art Peptide

Solvents for test peptides: Milli-Q water filtered through 0.22 μM filter; resistance—18.2 Mega Ohms

Phosphate buffered saline: pH 704 from Gibco (Cat. #100113-023)

20. mM sodium acetate buffer: pH 4.5 prepared with Milli-Q water and filtered through 0.45 μM filter.

Acetonitrile

Peptides: The test peptides included those of SEQ ID NOs: 2, 3, 4 & 7.

Peptide preparation: Test peptides were dissolved in either PBS or sodium acetate buffer at a concentration of 0.2 mg per ml. The purity of the test peptides was greater than 95%.

Method for testing stability: Stability of the peptides was examined at pH7.4 and at room temperature, 4° C. and 37° C. The amount of intact peptide remaining over time was compared with the initial intact amount. The quantification of the amount of peptide was based on peak height in an HPLC profile.

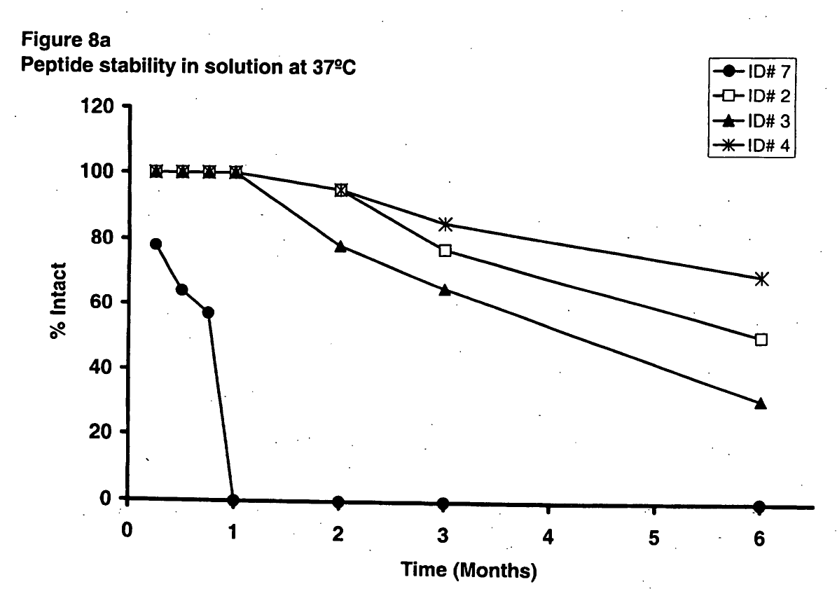

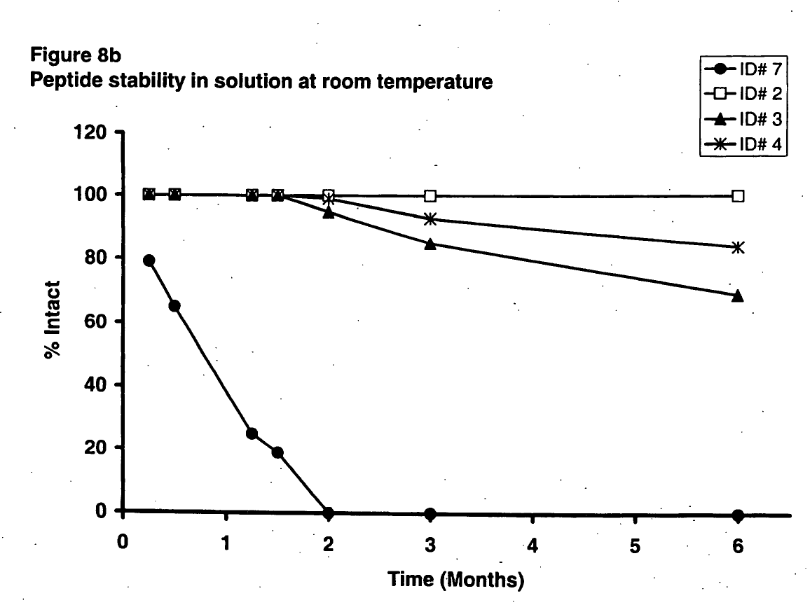

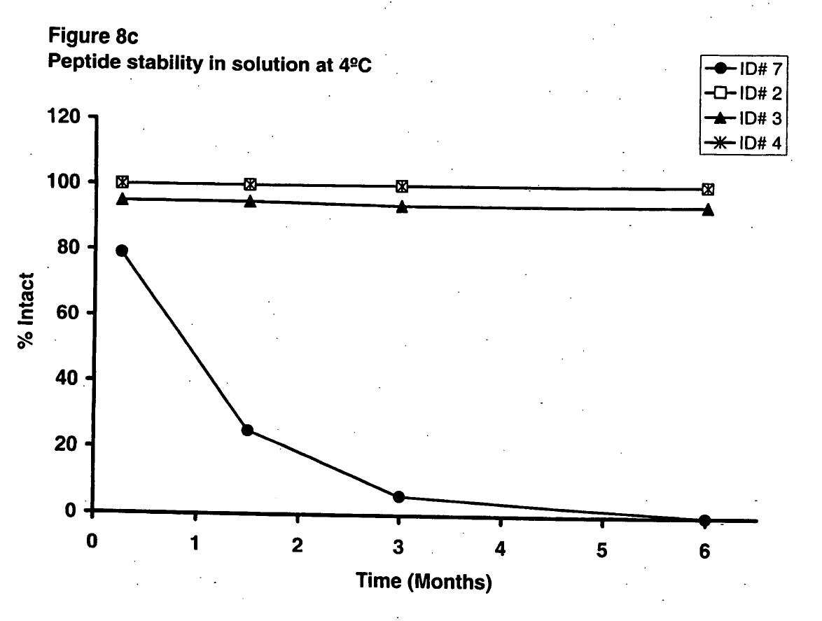

FIGS. 8(a), 8(b) and 8(c) illustrate the results of the stability of the four peptides tested. Peptides having SEQ ID NO:2 and 4 were the most stable. There was minimal degradation less than 5% in solution at pH 7.4 and at both 4° C. and at room temperature over a 6 month period SEQ ID NO:7 showed substantial degradation over three months under these conditions and it was totally degraded in 6 months time.

At 37° C., SEQ ID NO:7 showed substantial degradation over 3 weeks, but the other three peptides remained substantially intact. After three months all the peptides were significantly degraded. The stability of the peptides of SEQ ID NO's: 2 and 4 should enable a variety of routes of administration, including transdermal administration.

### Example 8

A Comparison of the In Vivo Activity of a Peptide According to the Invention Compared to a Prior Art Peptide

Female Sprague-Dawley rats, about 5 months old at the initiation of the procedure, were used. The rats were obtained from Vivarium of the University of Toronto, Mississauga, Ontario, Canada. The rats were divided into five groups each of the fourteen animals.

The rats were housed in pairs in standard shoebox cage in all groups. Humidity was maintained at 50%; temperature 22°; dark/light cycle of 12 hours. The rats were fed Standard Purina Rat Chow (unlimited daily supply) and tap water ad libitum.

The animals in four of the five groups were ovariectomized. In the remaining group, the animals underwent surgery without ovariectomy and served as a sham group.

Ovariectomies were performed under general isofluorane anesthesia. A 1.5 cm dorsal midline incision was made. The skin and subcutaneous tissue were separated by blunt dissection. A small incision was made on either side in the posterior abdominal muscle wall (approximately 0.5 cm) to expose the ovaries. The ovaries were removed together with the proximal ends of the Fallopian tubes after ligation of the ovarian arteries. The wounds in the abdominal muscle wall were not stitched. The dorsal skin wound was closed by three surgical wound clips. Tamgesic (0.2 ml) was given subcutaneously for the relief of pain immediately after the operation.

Body weight measurements were made by direct weighing with an electronic balance (to 0.01 g) and by DEXA measurement, using a Hologic 4500A instrument programmed for regional measurement for small animals. The animals were scanned supine under general isofluorane anesthesia. The whole body was scanned including the whole tail. The weights for the mineral content, the fat content and the lean body mass were summed to obtain total body weight.

Utility of DEXA for the Determination of Body Composition

DEXA measurements were evaluated as an indication of body composition. Through the attenuation of X-ray energy DEXA measurements provide an indication of the mass of various body tissue, fat and lean body mass (largely muscle mass). The sum of the measured masses represents total body mass. If this proposition is correct, then the total body mass measured by DEXA and measured directly by balance will be the same. Over the course of these experiments, paired measurements of the body weight were made using the two methods. The results obtained indicated that there is a close relationship between the DEXA measurements and the direct measurements (by balance) and consequently it was concluded that the body composition could be assessed using DEXA measurements.

Experimental Protocol

| | |
|---|---|
| Group 1 | Sham Control |
| Group 2 | OVX, PBS |
| Group 3 | OVX, Peptide, SEQ ID NO: 2 |
| Group 4 | OVX, Peptide, SEQ ID NO: 2 |
| Group 5 | OVX, Peptide, SEQ ID NO: 7 |

In Groups 3 and 4 the peptide of SEQ ID NO:2 was administered at doses of 300 and 800 nmoles per kg of body weight respectively. In Group 5, the peptide was administered of SEQ ID NO: 7 at a dose of 300 nmoles per kg of body weight. The test peptides were injected subcutaneously into the animals of Groups 3, 4 and 5 in 400 μl of 20 mM sodium acetate (ph4.5) daily, five days a week.

The animals in Group 1 were injected subcutaneously daily with 400 μl of 20 mM sodium acetate (pH4.5), 5 days per week. The animals in Group 2 were injected subcutaneously daily with 20 μl of PBS, 5 days per week.

Ovariectomies were performed at the age of 7 months. The ovariectomized animals were allowed to lose bone for two months following ovariectomy before the treatment was initiated. A baseline measurement was made immediately prior to operation. Another pretreatment baseline measurement was made at the beginning of treatment, i.e. two months after ovariectomy.

The results obtained three months after the initiation of treatment are reported below.

Effect of Ovariectomy on Body Weight

Ovariectomized rats gained weight more rapidly that intact rats, particularly during the first two months following the operation. The gain during this time period was about 100 g or more.

Effect of Peptide Treatment on Bone Mineral Content

An age-related gain in bone mineral content was observed for all groups of rats over the experimental period. The (BMC) gain was very pronounced for the first three months.

Ovariectomized rats appeared to experience a greater increase in bone mineral content with age than the intact rats. This appears to be in contrast with that observed in human beings, in which total body mineral content has been observed to decrease after menopause.

An attenuation of the increase in bone mineral content has been observed for rats treated with the peptide, for both intact and ovariectomized rats.

Bone Mineral Content and Body Weight

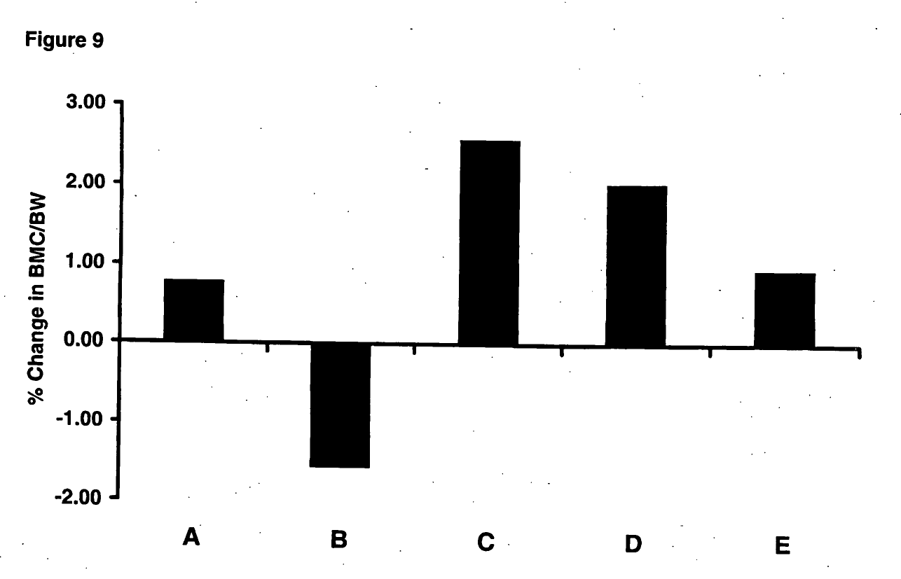

The percent change in the ratio of MBC/BW (as a function of time) was plotted, and is shown in FIG. 9.

As can be seen, there was a significant increase over the three month period in the BMC/BW ratio in the rats of Groups 3 and 4 treated with the peptide of SEQ ID NO:2 (Bars C and D), compared to the rats in Groups 1, 2 and 5 (Bars A, B and E). The replacement of the cysteine at position 9 in the prior art peptide of SEQ ID NO:7 by methionine had a positive effect on the ratio. It would be expected that an increase in the BMC/BW ratio would result in an improved load bearing capacity of the bone.

The peptides of the invention are useful in the treatment of bone related diseases, such as osteoporosis, or conditions requiring bone healing or repair.

The peptides of the invention can be administered directly (as by injection) or parenterally, such as by intravenous, subcutaneous, intramuscular, buccal, rectal, vaginal, intranasal or by aerosol administration. The therapeutic composition is preferably in the form of an aqueous solution, which is physiologically acceptable so that in addition to delivery of the desired peptide to the patient, the solution does not otherwise adversely affect the patient's electrolyte and volume balance. The aqueous medium for the therapeutic molecule thus may comprise, for example, normal physiological saline (0.9% NaCl, 0.15M), pH 7-7.4 or other pharmaceutically acceptable diluent.

Useful solutions for oral or parenteral administration may be prepared by any of the methods well known in the pharmaceutical art, described, for example, in “Remington's Pharmaceutical Sciences”, (Gennaro, A., ed.), Mack Pub., 1990. Formulations may include, for example, polyalkylene glycols such as polyethylene glycol, oils of vegetable origin, hydrogenated naphthalenes, and the like. Formulations for direct administration, in particular, may include glycerol and other compositions of high viscosity. Biocompatible, preferably bioresorbable polymers, including, for example, hyaluronic acid, collagen, tricalcium phosphate, polybutyrate, polylactide, polyglycolide and lactide/glycolide copolymers, may be useful excipients to control the release of the peptide in vivo.

Other potentially useful parenteral delivery systems for these therapeutic peptides include ethylene-vinyl acetate copolymer particles, osmotic pumps, implantable infusion systems, and liposomes. Formulations for inhalation administration may contain as excipients, for example, lactose, or may be aqueous solutions containing, for example, polyoxyethylene-9-lauryl ether, glycocholate and deoxycholate, or oily solutions for administration in the form of nasal drops, or as a gel to be applied intransally.

The therapeutic peptides may be administered alone or in combination with other molecules known to aid tissue growth or repair, i.e., molecules capable of tissue repair and regeneration and/or inhibiting inflammation. Examples of useful cofactors for stimulating bone tissue growth in osteoporotic individuals, for example, include but are not limited to, vitamin D₃, calcitonin, prostaglandins, parathyroid hormone, dexamethasone, estrogen and IGF-I or IGF-II.

The therapeutic peptides further can be formulated into pharmaceutical compositions by admixture with pharmaceutically acceptable nontoxic excipients and carriers. As noted above, such compositions may be prepared for parenteral administration, particularly in the form of liquid solutions or suspensions; for oral administration, particularly in the form of tablets or capsules; or intranasally, particularly in the form of powders, nasal drops or aerosols. Where adhesion to a tissue surface is desired the composition may include the biosynthetic construct dispersed in a fibrinogen-thrombin composition or other bioadhesive such as is disclosed, for example in PCT US91/09275. The composition then may be painted, sprayed or otherwise applied to the desired tissue surface.

The compositions can be formulated for parenteral or oral administration to humans or other mammals in therapeutically effective amounts, e.g., amounts which provide appropriate concentrations of the molecule to target tissue for a time sufficient to induce the desired effect.

It is contemplated also that a peptide of the invention may exhibit a high level of activity in vivo when combined with carrier matrices i.e., insoluble polymer matrices. Carrier matrices include those that are xenogenic, allogenic or autogenic in nature. It is contemplated that synthetic materials comprising polylactic acid, polyglycolic acid, polybutyric acid, derivatives and copolymers thereof may also be used to generate suitable carrier matrices. Synthetic and naturally derived matrix materials, their preparation, methods for formulating them, and methods of administration are well known in the art and so are not discussed in detailed. (see for example, U.S. Pat. No. 5,266,683.)

As will be appreciated by those skilled in the art, the concentration of the peptides described in a therapeutic composition will vary depending upon a number of factors, including the dosage of the drug to be administered, the chemical characteristics (e.g., hydrophobicity) of the compounds employed, and the route of administration. The preferred dosage of drug to be administered also is likely to depend on such variables as the type and extent of a disease, tissue loss or defect, the overall health status of the particular patient, the relative biological efficacy of the compound selected, the formulation of the compound, the presence and types of excipients in the formulation, and the route of administration. In general terms, the therapeutic molecules of this invention may be provided to and individual where typical doses range from about 10 ng/kg to about 1 g/kg of body weight per day; with a preferred dose range being from about 0.1 mg/kg to 100 mg/kg of body weight.

The present invention also includes diagnostic applications based on measuring the presence and extent of a peptide of the invention in biological extracts (or samples) taken from a subject being treated for osteoporosis or other condition for which the peptide has effect. In this way, the level of the peptide being administered can be monitored in an individual, be it by a doctor, clinician, or even the subject, provided with a suitable diagnostic kit. A kit can include an antibody to the particular peptide being administered and which binds to it. The antibodies can be generated using standard procedures known in the art, as described in WO 94/20614 and U.S. Pat. No. 6,475,753, for example.

The antibody can be linked to or conjugated with any of several well known reporter systems set up to indicate positively, binding of the peptide to the antibody. Well known reporter systems include radio immunoassays (RIAs) or immunoradiometric assays (IRMAs). Alternatively, an enzyme-linked immunosorbent assay (ELISA) would have in common with RIAs and IRMAs a relatively high degree of sensitivity, but would generally not rely upon the use of radioisotopes. A visually detectable substance may be produced or one detectable in a spectrophotometer. An assay relying upon fluorescence of a substance bound by the enzyme being assayed could be used. It will be appreciated that there are several reporter systems which may be used, according to the present invention, to detect the presence of a particular peptide. With standardized sample collection and treatment, peptide presence below or above a threshold amount in blood serum, urine or other sample can be determined. This in turn can be used in determining dosage levels, administration frequency, and other aspects of a treatment regimen.

Also included in the invention is the use of the peptides in competitive assays to identify or quantify molecules having receptor binding characteristics corresponding to those of the peptides. The peptides may be labeled, optionally with a radioisotope. A competitive assay can identify both antagonists and agonist of the relevant receptor.

The terms “comprising” or “comprises” are used herein in an open-ended sense unless the context would clearly dictate otherwise. Thus, a “feature comprising A and B” is a feature that includes elements A and B, but may also include other elements.

Although various examples of combined elements of the invention have been described, it will also be understood that these are not intended to be exhaustive and features of one embodiment may be combined with those of another, and such other combinations are contemplated to be within the scope of the invention disclosed herein.

All documents mentioned herein are incorporated into this specification in their entirety as though their entire contents were reproduced here. Applicant reserves the right to incorporate into this specification, or any application claiming priority from it, any part of any document referred to herein at any time during pendency of such application.

From the foregoing description, one skilled in the art can easily ascertain the essential characteristics of this invention, and without departing from the spirit and scope thereof can make various changes and modifications of the invention to adapt it to various usages and conditions.

1. A compound having bone stimulatory activity, the compound comprising a peptide having an amino acid sequence of Formula I: 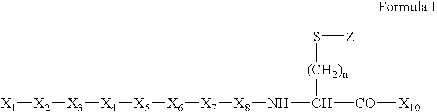
in which:
X₁ and X₁₀ are positively charged polar amino acids;
X₄ and X₈ are negatively charged polar amino acids;
X₅ is an aromatic amino acid;
X₂, X₃, X₆ and X₇ are non polar neutral amino acids or uncharged polar amino acids;
Z represents a blocking group; and n is an integer from 1 to 3.

2. A compound of claim 1, in which each of X₁ and X₁₀ is independently selected from the group of arginine and lysine; each of X₂, X₃, X₆ and X₇ is independently selected from the group of threonine, valine, serine, alanine or glutamine; X₅ is histidine or phenylalanine; each of X₄ and X₈ is aspartic acid or glutamic acid; and Z is a substituted or unsubstituted alkyl, carboxyalkyl or carboxyamidoalkyl group.

3. A compound of claim 1, in which Z is selected from the group consisting of a lower alkyl group, carboxyloweralkyl or carboxyamidoloweralkyl.

4. A compound of claim 3, in which the alkyl group is methyl or ethyl and n is 1 or 2.

5. A compound of claim 2 in which the alkyl group of Z is methyl.

6. A peptide with bone stimulatory activity comprising an amino acid sequence containing 10-amino acids selected from the group consisting of peptides of the following Formula Ia:
K T Q E F T A E X₉ K
R T Q E F T A E X₉ K
R T Q E H T A E X₉ K
K T Q E H T A E X₉ K Formula Ia
in which X₉ is methionine or a modified methionine or a modified cysteine.

7. A peptide of claim 6, in which X₉, when a modified methionine or a modified cysteine, is represented by the formula: 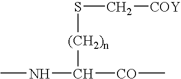
wherein Y represents a hydroxyl, alkoxy or amino group; and n is an integer from 1-3.

8. A peptide of claim 7 in which n is 1 or 2.

9. A peptide of claim 7 in which at least one of the C-terminus of the peptide or the N-terminus of the peptide includes a protecting group.

10. A peptide of claim 9, wherein the protecting group of the N-terminus is an acetyl group, and the protecting group of the C-terminus is an amino group.

11. A peptide having the amino acid sequence identified as SEQ ID NO:1, wherein the N-terminus is optionally protected with an acetyl group, and the C-terminus optionally protected with an amino group.

12. A peptide having the amino acid sequence identified as SEQ ID NO:2, wherein the N-terminus is optionally protected with an acetyl group, and the C-terminus optionally protected with an amino group.

13. A peptide having the amino acid sequence identified as SEQ ID NO:3, wherein the N-terminus is optionally protected with an acetyl group, and the C-terminus optionally protected with an amino group.

14. A peptide having the amino acid sequence identified as SEQ ID NO:4, wherein the N-terminus is optionally protected with an acetyl group, and the C-terminus optionally protected with an amino group.

15. A peptide having the amino acid sequence identified as SEQ ID NO:5, wherein the N-terminus is optionally protected with an acetyl group, and the C-terminus optionally protected with an amino group.

16. A peptide having the amino acid sequence identified as SEQ ID NO:6, wherein the N-terminus is optionally protected with an acetyl group, and the C-terminus optionally protected with an amino group.

17. A method of stimulating bone growth in a mammal comprising administering to the mammal an effective amount of a compound according to claim 1.

18. A method of treating osteoporosis in a mammal comprising administering to a mammal a therapeutically effective amount of a compound according to claim 1.

19. A pharmaceutical composition comprising a pharmaceutically acceptable carrier and a therapeutically acceptable amount of a compound according to claim 1.

20-22. (canceled)

23. A compound of claim 2 in which Z is selected from the group consisting of a lower alkyl group, carboxyloweralkyl or carboxyamidoloweralkyl.
